(19) United States
(12) Patent Application Publication
(10) Pub. No.: US 2024/0362515 A1
(43) Pub. Date: Oct. 31, 2024

(54) Anonemis Research Software Hard Drive Protocol Policy Processes 2303

(71) Applicant: Renee Simenona Martinez, Beverly Hills, CA (US)

(72) Inventor: Renee Simenona Martinez, Beverly Hills, CA (US)

(73) Assignee: Renee Simenona Martinez, Beverly Hills, CA (US)

(21) Appl. No.: 18/141,410

(22) Filed: Apr. 29, 2023

(51) Int. Cl. G06N 10/60; G16H 10/20

(52) U.S. Cl. CPC G06N 10/60; G16H 10/20

(57) ABSTRACT

In a quantum sequence triplication embodiment with quantum operation supremacy, the algorithmic software process and system database are utilized to critically verify information by Quantum Sequence Triplication, which utilizes three qubit bits to achieve quantum supremacy without gray area bias and enables quantum supremacy in the absence of gray area bias.

Neurological Profiles For Market Matching And Stimulus Presentation/Ser. No. 16/561,980
Pattern-based Classification/Ser. No. 16/912,009.
Unusual Score Generators For A Neuro-linguistic Behavioral Recognition System/Ser. No. 17/164,210
SYSTEM AND METHOD FOR GENERATING CURATED INTERVENTIONS IN RESPONSE TO PATIENT BEHAVIOR/Ser. No. 17/213,164
Generating Ratings Predictions Using Neuro-response Data/Ser. No. 16/152,980.
System, Method, And Computer Program Product For Analog And Mix-signal Circuit Placement/Ser. No. 16/238,274.
USER BEHAVORIAL ANALYTICS FOR SECURITY ANOMALY DETECTION IN INDUSTRIAL CONTROL SYSTEMS/Ser. No. 17/442,834.
Span Pointer Networks For Non-autoregressive Task-oriented Semantic Parsing For Assistant Systems Ser. No. 17/525,510
Personalized Content Delivery Using Neuro-response Priming Data/Ser. No. 16/193,930
ENHANCED RISK ASSESSMENT/Ser. No. 17/039,009
Training A Machine Learning System For ADC Compensation/Ser. No. 16/933,617.
Efficient Memory Leak Detection In Database Systems/Ser. No. 18/169,271
Sharing Probabilities For User Traits/Ser. No. 15/132,192.
Compositions And Methods For Detecting Predisposition To Cardiovascular Disease/Ser. No. 17/857,723
Medical Image Analysis Method, Medical Image Analysis Device, And Medical Image Analysis System/Ser. No. 17/515,466
Methods For Diagnosing An Autistic Spectrum Disorder/Ser. No. 16/967,361.
Health And Wellness Management Technology/Ser. No. 16/891,403.
Sensor Device And Methods/Ser. No. 16/471,552.
Systems, Methods And Apparatuses Of Digital Assistants In An Augmented Reality Environment And Local Determination Of Virtual Object Placement And Apparatuses Of Single Or Multi-directional Lens As Portals Between A Physical World And A Digital World Component Of The Augmented Reality Environment/Ser. No. 16/853,815.
NEUROLOGICAL REHABILITATION AND TRAINING METHOD UTILIZING OCULOMOTOR, VISUAL AND/OR VESTIBULAR REHABILITATION TASKS ON SUBJECTS WITH PHARMACOLOGICALLY INDUCED NEUROPLASTICITY/Ser. No. 17/316,077.
Interactive And Analytical System That Provides A Dynamic Tool For Therapies To Prevent And Cure Dementia-related Diseases/Ser. No. 17/676,562.
Molecule Counting Of Methylated Cell-free DNA For Treatment Monitoring/Ser. No. 18/187,578.
Management Of Connected Sensor Devices/Ser. No. 17/390,811
Machine Learning Model To Determine A Proper Subset Of A Multimedia File For A Viewer Based On Their Interest/Ser. No. 17/521,668
Rule Creation For Code Analysis/Ser. No. 17/850,583.
Methods, Systems, And Computer Readable Media For Suppressing Cookie Based Advertisement Retargeting/Ser. No. 15/374,653.
Technique For Alignment Of A Mobile Device Orientation Sensor With The Earth's Coordinate System/Ser. No. 16/298,410
Mapping Non-attached Subnetworks/Ser. No. 16/692,034
Batch Normalization For Replicated Layers Of Neural Network/Ser. No. 16/923,006
Error And Manipulation Resistant Search Technology/Ser. No. 16/886,986
Batch Normalization For Replicated Layers Of Neural Network/Ser. No. 16/923,006

### STATEMENT REGARDING FEDERALLY SPONSORED RESEARCH OR DEVELOPMENT

N/A

### THE NAMES OF THE PARTIES TO A JOINT RESEARCH AGREEMENT

Renee Simenona Martinez, Anonemis Research, and Scigenity Technology

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Standard Application Requirements.

### STATEMENT REGARDING PRIOR DISCLOSURES BY THE INVENTOR OR A JOINT INVENTOR

Invention has not been disclosed.

### BACKGROUND OF THE INVENTION:

### (1) Field of the Invention

Quantum Sequence Triplication with Quantum Operation Supremacy is an embodiment comprising of an algorithmic software process and system database which data source references are critically verified information by Quantum Sequence Triplication, where the use of three qubit bits to achieve Quantum Supremacy without grey area bias. Qubit 1 and 2, are encoded using the Quantum Operation Supremacy methodology, except for the data being opposing information data. Finally, qubit 3, is the output of the data calculation process of opposing outcomes to determine the sum data devoid of grey area but most importantly, fact checked. This system database uses current and real-time information which is vetted within the system queue for verification by both automated algorithms and operator oversight. Further objects, features, advantages and properties of this software and methodology according to the present application will become apparent from the detailed description.

### (2) Description of Related Art Including Information Disclosed Under 37 CFR 1.97 and 1.98

N/A

### BRIEF SUMMARY OF THE INVENTION

In the following detailed description, the software process according to the teachings for this application in the form of technical science will be described by the embodiments. It should be noted that the teachings of this application can also be used in any other sectors such as: telecommunications, automotives, transliteration development, chip technology, communications, data analysis, behavioral science, transportation, medicine, nuclear science, space technology, defense, quantum approximation, data algorithm structure, mechanical engineering and any software devices offering access to information.

The first embodiment comprises of a system that works by implementing a specialized rapid algorithm process which correlates data in real-time and produces predictive data which is precisely relevant to the case studies and content is illustrated in FIG. 1. The data analyzes the content from various sources to provide the user with a better advantage in any given circumstance. In Quantum Sequence Triplication, 3 qubit bits are used to achieve quantum supremacy without grey area bias. Qubit 1 and 2, are encoded using the Quantum Operation Supremacy methodology, except for the data being opposing information and data.

Finally, qubit 3, is the output of the data calculation process of opposing outcomes to determine the sum data devoid of grey area but most importantly, fact checked.

The technology is intended for military strategy and defense, medicine and medical development without error, and the groundwork for quantum supremacy.

### BRIEF DESCRIPTION OF THE SEVERAL VIEWS OF THE DRAWING(S)

In the following detailed portion of the present description, the teachings of the present application will be explained in more detail with reference to the example embodiments shown in the drawings, in which:

FIG. 1. AR Quantum Sequence Triplication Algorithm with Quantum Operation Supremacy

FIG. 2. AR Quantum Telecommunication Tiers

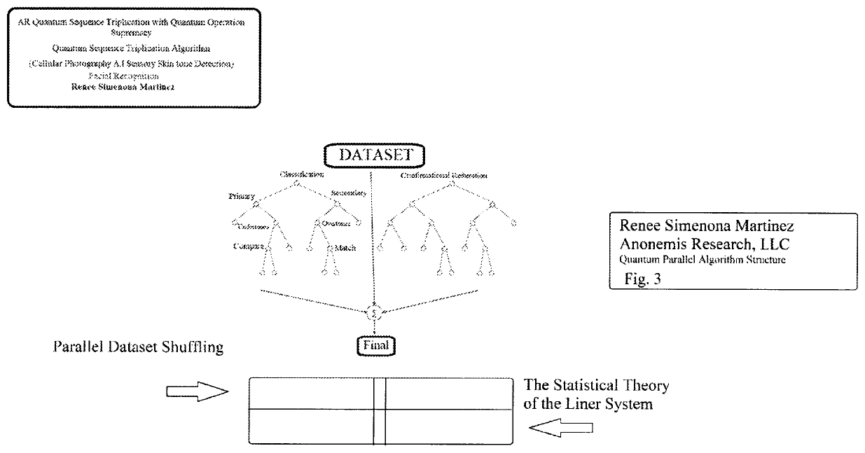

FIG. 3. AR Quantum Parallel Algorithm Structure/AR Quantum Sequence Triplication Algorithm with Quantum Operation Supremacy

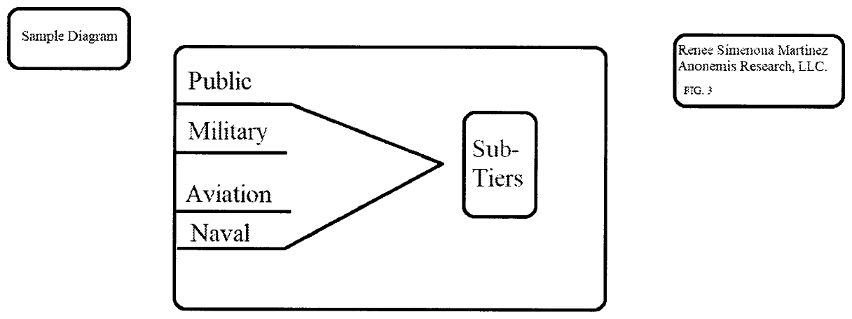

FIG. 4. AR Cognitive Conscious Recovery System

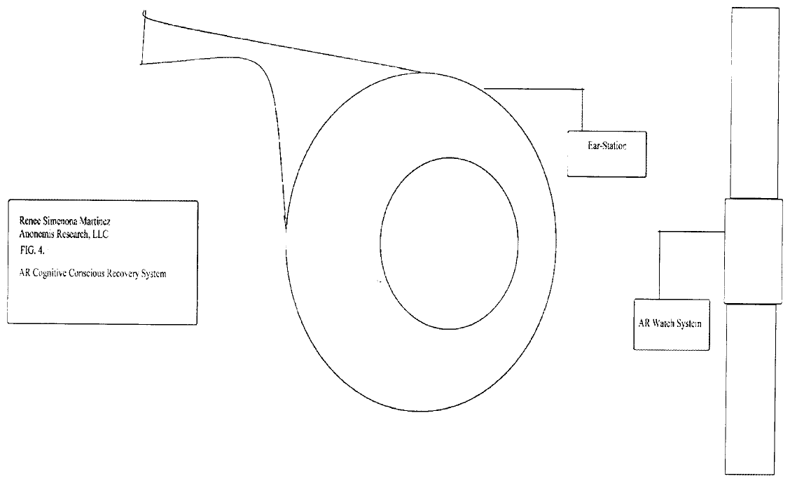

### DETAILED DESCRIPTION OF THE INVENTION

In the following detailed description, the software process according to the teachings for this application in the form of technical science will be described by the embodiments. It should be noted that the teachings of this application can also be used in any other sectors such as: telecommunications, automotives, transliteration development, chip technology, communications, data analysis, behavioral science, transportation, medicine, nuclear science, space technology, defense, quantum approximation, data algorithm structure, mechanical engineering and any software devices offering access to information.

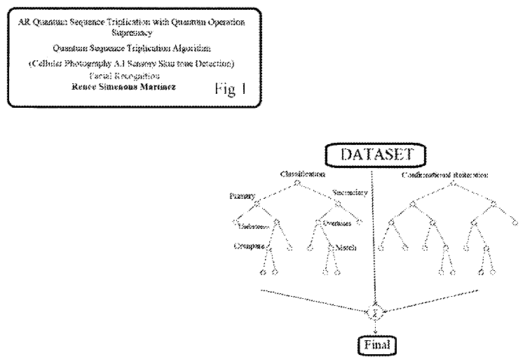

The first embodiment comprises of a system that works by implementing a specialized rapid algorithm process which correlates data in real-time and produces predictive data which is precisely relevant to the case studies and content is illustrated in FIG. 1. The data analyzes the content from various sources to provide the user with a better advantage in any given circumstance. In Quantum Sequence Triplication, 3 qubit bits are used to achieve quantum supremacy without grey area bias. Qubit 1 and 2, are encoded using the Quantum Operation Supremacy methodology, except for the data being opposing information and data. Finally, qubit 3, is the output of the data calculation process of opposing outcomes to determine the sum data devoid of grey area but most importantly, fact checked.

The technology is intended for military strategy and defense, medicine and medical development without error, and the groundwork for quantum supremacy.

The use of traditional binary single qubit output methodology is the baseline for this operation and in the alteration regarding quantum operation. The system works by implementing artificial qubits to source and produce calculation data for equation execution. The data is stored and categorized into more ridged data columns to eliminate error and to achieve simplification. Sorting data by patterned repetitions in the level of importance relevance for desired outcome. This rapid data shuffling and detecting repetition sources regarding patterns can assist solving complex cryptology.

The operating system for the user interface includes several of the elements regarding an interactive precision experience.

The invention claimed is:

1. An embodiment of the present application comprises of a quantum sequence triplication embodiment with quantum operation supremacy, the algorithmic software process and system database are utilized to critically verify information by Quantum Sequence Triplication, where three qubit bits are used to achieve quantum supremacy without gray area bias.

1. A software process and methodology according to claim 1, wherein compromising of a specialized encrypted algorithm with database for military, medical personnel and researchers in various tactical areas have access to stated search engine to determine computation results in Macotrophic Forensic Magnetic Resonance Imaging produces a detailed anatomical flesh image in addition to a three-dimensional detailed anatomical image which allows the administrator to see in detail from every angle but provides an innovative procedural view of an in-depth perspective of both internal imaging and a flesh examination.

2. A software process and methodology according to claim 1, wherein compromising of a specialized encrypted database for military personnel and researchers with security clearances regarding various tactical areas have access to stated search engine to determine computation results in Forensic Indicative Imaging is a system infrastructure and device designed to implement full body scanning to detect small injuries to significant bodily harm with precision with 3D viewing such as: detect depressions and entry wounds and determine possible assault items by using an A.I database which detailed ridging and designs ideocracies of knives, axes, and everyday items.

3. A software process and methodology according to claim 1, wherein compromising of a specialized encrypted database for military personnel and researchers with security clearances regarding various tactical areas have access to stated search engine to determine computation results in Forensic Indicative Molding, a system infrastructure and device designed to implement full body spray molding within a podlike design, which will hold the evidence in place without disruption, alteration when scanned thoroughly with maco-lense to detect fingerprints, body disruptions, chemicals, and fluids by comparing intelligence imputation details from database.

4. A software process and methodology according to claim 1, wherein compromising of a specialized encrypted database for military personnel and researchers with security clearances regarding various tactical areas have access to stated search engine to determine computation results in Aqua Pura Cell-Cell Recognition Coagulation In Remain Recovery is the detection of body secretions during the recovery of the body in large expanses of water, such as hormones, sweat, and fluids from the organs which is commonly known that adhesive glycoproteins such as entactin's (or nidogens), fibronectins, fibrinogens, thrombospondins, vitronectins, nephronectins, and laminins are responsible for cell adhesion. Thus, Biochemically and structurally, glycoproteins play a vital role. The function of these molecules is to allow cells to recognize another cell as familiar or foreign, which is known as cell-cell recognition. Furthermore, they facilitate the attachment and binding of cells to one another, which is known as cell-cell recognition.

5. A software process and methodology according to claim 1, wherein compromising of a specialized encrypted database for military personnel and researchers with security clearances regarding various tactical areas have access to stated search engine to determine computation results in M-B Pheno Forensic Software and Methodology Process is Mandatory biometrics and Micotrophic Forensic Lens scanning upon convictions and incarceration.
